(19) United States
(12) Patent Application Publication
(10) Pub. No.: US 2021/0369893 A1
(43) Pub. Date: Dec. 2, 2021

(54) SANITATION ASSEMBLY

(71) Applicant: R Zero, Inc., Ridgeland, MS (US)

(72) Inventor: Frederick Kennedy Sones, Madison, MS (US)

(73) Assignee: R Zero, Inc., Ridgeland, MS (US)

(21) Appl. No.: 17/332,711

(22) Filed: May 27, 2021

Related U.S. Application Data
(60) Provisional application No. 63/030,511, filed on May 27, 2020.

(51) Int. Cl. A61L 2/10; A61L 2/26; A61L 2/20

(52) U.S. Cl. CPC A61L 2/10; A61L 2/26; A61L 2202/14; A61L 2202/25; A61L 2202/11; A61L 2/202

(57) ABSTRACT

A sanitation assembly for sanitizing and/or sterilizing a space.

### CROSS-REFERENCE TO RELATED APPLICATION

This application claims priority to U.S. Provisional Application No. 63/030,511 filed May 27, 2020, which is incorporated herein by reference in its entirety.

### FIELD OF THE DISCLOSURE

The present disclosure is generally directed toward a sanitation assembly, specifically a sanitation assembly for sterilizing rooms.

### BACKGROUND OF THE DISCLOSURE

In some instances, sanitation and/or sterilization of a space or facility may be useful. The use of UV-C lights for facility sterilization is an emerging technology.

### SUMMARY OF THE DISCLOSURE

A sanitation assembly includes one of a UV-C LED and an ozone generator positioned within a space and a control system configured to selectively actuate the one of a UV-C LED and an ozone generator, wherein the control system is configured as a remote based control system.

A sanitation assembly includes one of a UV-C LED and an ozone generator positioned within a space, a control system configured to selectively actuate the one of a UV-C LED and an ozone generator, and a sensor in communication with the control system.

A UV-C light system includes one or more UV-C LED that fit in standard light fixtures and uses various control systems to ensure that irradiation of a space does not occur in an uncontrolled manner.

The claimed system prevents individuals from being exposed to UV-C light, ozone, or other sanitation and/or sterilization methods that are traditionally used for facility sanitation/sterilization.

### DETAILED DESCRIPTION

The following detailed description is presented to enable any person skilled in the art to make and use the disclosure. For purposes of explanation, specific details are set forth to provide a thorough understanding of the present disclosure. However, it will be apparent to one skilled in the art that these specific details are not required to practice the disclosure. Descriptions of specific applications are provided only as representative examples. Various modifications to the preferred embodiments will be readily apparent to one skilled in the art, and the general principles defined herein may be applied to other embodiments and applications without departing from the scope of the disclosure. The present disclosure is not intended to be limited to the embodiments shown but is to be accorded the widest possible scope consistent with the principles and features disclosed herein.

As disclosed herein, a sanitation and/or sterilization assembly includes at least one light source and/or at least one sanitation and/or sterilization component. For example, the assembly may include a UV-C light emitting diode (LED) and/or an ozone generator. However, it is contemplated that the assembly may include only the UV-C LED or only the ozone generator. It is also contemplated that the assembly may include multiple UV-C LEDs. It is further contemplated that the assembly may include other components configured to provide sanitation and/or sterilization of a space (e.g., a room or facility). Where the assembly includes a UV-C LED, the UV-C LED may be configured to fit in standard light fixtures. Alternatively, the UV-C LED may be configured to be otherwise operably coupled with an electrical grid of a facility or any other power source.

The assembly may be configured to include a control system to ensure that irradiation of a space does not occur in an uncontrolled manner. In various examples, the control system may include a photocell that engages irradiation after a certain time of being in the dark also after a certain time of day. In other examples, the control system may include a motion sensor that utilizes machine learning to sense when the room is empty to irradiate the room. In still other examples, the control system may include a suite or plurality of UV-C LEDs connected to another power regulator. This regulator, as opposed to other exemplary control systems discussed previously, may be connected to Wi-Fi. In still other examples, the control system may be configured to actuated using remote based scheduling and/or remote connections and/or controls. For example, an application that is accessible to building owners and maintenance personnel may be configured to allow for the assembly to be controlled both manually and at on a pre-set schedule. In another example, the control system may be configured to be controlled via a web-based control. It is contemplated that the control system may be configured to include any combination of remote based controls without departing from the scope of the present disclosure.

Furthermore, the control system may consist of the ability to mitigate the problem of using UV disinfection in instances which may involve immobile or unconscious people in the room with the device. For example, an easily measurable and inescapable evidence of human activity is thermal activity, as the human body temperature is roughly 98.6 F, well above ambient room temperature. Using thermal cameras, it may be possible to detect live individuals in a room regardless of movement. Machine learning may be employed to filter out noise in environments without stable temperatures and with heat-generating devices such as refrigerators. The system also may further include one or more sensors configured to measure other factors in a space, facility, or room. For example, the control system may include CO2 sensors or sound sensors.

One assembly will be needed per room and will hook into the existing interne of things (“TOT”) framework. Each assembly may be connected with one or more other assemblies by the standard TOT connections (Wi-Fi, Bluetooth, mesh, etc.). In various examples, the assemblies may be configured to be actuated in sequence (e.g., one after another) via a timer or other control system. In other examples, the assemblies may be configured to be remotely actuated in tandem or individually without departing from the scope of the present disclosure.

The control system may be configured to actuate the UV-C LED based on a wavelength of the light emitted from the UV-C LED (within a range of about 200 to about 280 nm) or based on wattage/time (“dosage”), which is variable depending on what the system is configured to target. For example, Legionella pneumophila may be killed by X watts (at surface)×X seconds (at surface) whereas a Rotavirus would need Y watts (at surface)×Y seconds (at surface). In various examples, a UV-C “dose” may be measured in joules per inch squared (J/in2) and UV-C intensity may be measured in measured in milliwatts per inch squared (mW/in2). The control system may be configured to control and tuned one or more of these variables. The variable may be based on a logarithmic reduction of whatever microorganism(s) are targeted.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the disclosure.

The disclosure has been described with reference to various specific and preferred embodiments and techniques. However, it should be understood that many variations and modifications may be made while remaining within the spirit and scope of the disclosure. It will be apparent to one of ordinary skill in the art that methods, devices, device elements, materials, procedures and techniques other than those specifically described herein can be applied to the practice of the disclosure as broadly disclosed herein without resort to undue experimentation. All art-known functional equivalents of methods, devices, device elements, materials, procedures and techniques described herein are intended to be encompassed by this disclosure. Whenever a range is disclosed, all subranges and individual values are intended to be encompassed. This disclosure is not to be limited by the embodiments disclosed, including any shown in the drawings or exemplified in the specification, which are given by way of example and not of limitation.

While the disclosure has been described with respect to a limited number of embodiments, those skilled in the art, having benefit of this disclosure, will appreciate that other embodiments can be devised which do not depart from the scope of the disclosure as disclosed herein. Accordingly, the scope of the disclosure should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

We claim:

1. A UV-C light room sterilization system comprising of LED of UV-C that fit in standard light fixtures.

2. The system of claim 1 further comprising a photocell that engages irradiation after a certain time-period of being in the dark and after a certain time of day.

3. The system of claim 1 further comprising a motion sensor that utilizes machine learning to sense when the room is empty to irradiate the room.

4. The system of claim 1 further comprising a suite of UV disinfection lights connected to another Wi-Fi connected power regulator.

5. The system of claim 1 further comprising various combinations of UV disinfection lights to measure other factors such as CO2 readings in the room and sound.

6. A sanitation assembly comprising:
one of a UV-C LED and an ozone generator positioned within a space;
a control system configured to selectively actuate the one of a UV-C LED and an ozone generator; and
a sensor in communication with the control system.

7. A sanitation assembly comprising:
one of a UV-C LED and an ozone generator positioned within a space; and
a control system configured to selectively actuate the one of a UV-C LED and an ozone generator, wherein the control system is configured as a remote based control system.
